(19) United States
(12) Patent Application Publication
(10) Pub. No.: US 2015/0140114 A1
(43) Pub. Date: May 21, 2015

(54) SKIN TREATMENT PROTOCOL UTILIZING AMNIOTIC SOLUTION

(71) Applicant: John R. SASKO, Delray Beach, FL (US)

(72) Inventor: John R. SASKO, Delray Beach, FL (US)

(21) Appl. No.: 14/546,034

(22) Filed: Nov. 18, 2014

Related U.S. Application Data
(60) Provisional application No. 61/905,897, filed on Nov. 19, 2013.

(51) Int. Cl. A61K 35/50; A61K 9/00

(52) U.S. Cl. CPC A61K 35/50; A61K 9/0014
USPC 424/528; 424/582

(57) ABSTRACT

A skin treatment method, including: performing a skin treatment procedure on the skin of a patient, wherein the skin treatment procedure includes one of a chemical peel procedure, a dermabrasion procedure, a micro-needling procedure, and a laser procedure; and subsequently, applying a preparation including one or more of amniotic fluid and amniotic membrane material to the treated skin of the patient to promote healing and collagen rejuvenation, wherein the preparation including the one or more of amniotic fluid and amniotic membrane material is prepared from one or more of amniotic fluid, a concentrated amniotic membrane solution, and an amniotic membrane powder. The preparation comprising the one or more of amniotic fluid and amniotic membrane material also includes sterile water or saline. The treated skin of the patent preferably includes one or more of the skin of the face and the skin of the neck.

### CROSS-REFERENCE TO RELATED APPLICATION(S)

The present patent application/patent claims the benefit of priority of co-pending U.S. Provisional Patent Application No. 61/905,897, filed on Nov. 19, 2013, and entitled “SKIN TREATMENT PROTOCOL UTILIZING AMNIOTIC SOLUTION,” the contents of which are included in full by reference herein.

### FIELD OF THE INVENTION

The present invention relates generally to a medical/cosmetic skin treatment procedure. More specifically, the present invention relates to a medical/cosmetic skin treatment procedure that utilizes an amniotic solution derived from the placenta or manufactured from amniotic membrane or the like that promotes quicker healing and collagen generation, making the skin healthier and more youthful in appearance.

### BACKGROUND OF THE INVENTION

A variety of medical/cosmetic skin treatment procedures exist for modifying the surface and quality of the skin, especially that of the face and neck. These skin treatment procedures may be performed in a medical office, a pseudo-medical office, and/or a spa or salon.

Aging, genetics, sun exposure, and lifestyle—including nutrition, smoking, and alcohol consumption may all contribute to facial wrinkling, discoloration, and collagen loss. Such conditions may be treated using a skin resurfacing procedure. Skin resurfacing procedures all involve “injuring” the skin and allowing a healing process to take place, resulting in healthier looking skin emerging. This may be enhanced by the post-procedure application of various masks, lotions, and/or solutions, typically containing plant extracts and/or other natural or synthetic compounds or chemicals that promote healing and collagen generation, making the skin healthier and more youthful in appearance. In addition, some are applying platelet-rich plasma (PRP) derived from a patient's own blood.

Chemical peels involve the application of a caustic solution, dermabrasion utilizes a high-speed abrasive rotary wheel or micro-needles, and laser resurfacing utilizes a laser beam to “injure” the skin.

Chemicals peels may use mild chemicals, such as glycolic acid or the like, to create very superficial changes in the skin, or more harsh chemicals, such as phenol or the like, to create more profound changes in the skin. As the potential for dramatic changes increases, so does the potential risk of permanent skin lightening or scarring. The chemicals are applied to the skin in the desired areas, and may be “neutralized” after an appropriate amount of time. The depth of the peeling action of the chemicals is determined by which chemicals are used, how long they remain on the skin, and whether they are applied lightly or rubbed more vigorously into the skin.

Chemical peels using alpha hydroxy acids (AHAs), such as glycolic acid or the like are the most mild. A single treatment with an AHA gives the skin a fresher, healthier appearance and a radiant glow. Repeated treatments may help to further improve the texture of the skin. No anesthesia or sedation is needed for this type of chemical peel, which produces only a mild tingling or stinging sensation. The post-procedure application of various masks, lotions, and/or solutions, typically containing plant extracts and/or other natural or synthetic compounds or chemicals that promote healing and collagen generation is possible.

Chemical peels using trichloracetic acid (TCA) are also common for treating sun damage. Milder chemical peels using TCA may be repeated frequently in order to achieve cumulative effects, or may be used to achieve a medium or deep chemical peel, depending on the acid concentration and manner of application. Again, the post-procedure application of various masks, lotions, and/or solutions, typically containing plant extracts and/or other natural or synthetic compounds or chemicals that promote healing and collagen generation is possible.

Chemical peels using phenol are common for treating particularly rough and sun-damaged facial skin, for example. However, phenol may cause excessive damage to the skin and result in bleaching, and is typically not used on the neck or other parts of the body. Phenol buffers may be used to create a “buffered” or milder solution, and may allow for greater flexibility in its use. Again, the post-procedure application of various masks, lotions, and/or solutions, typically containing plant extracts and/or other natural or synthetic compounds or chemicals that promote healing and collagen generation is possible.

Dermabrasion uses a high-speed abrasive rotary wheel or micro-needles to abrade or puncture the skin, removing its upper layers. Dermabrasion is sometimes selected for the treatment of facial scars and the like. Dermabrasion often requires some form of anesthesia, whether local or sedation. Again, the post-procedure application of various masks, lotions, and/or solutions, typically containing plant extracts and/or other natural or synthetic compounds or chemicals that promote healing and collagen generation is possible.

Laser resurfacing either creates a uniform injury to the skin, similar to a deeper chemical peel or dermabrasion, or “drills” tiny holes into the deeper layers of the skin in “fractional resurfacing,” where collagen contracts between the tiny holes. Laser resurfacing typically requires anesthesia. Depending on the laser treatment chosen and the area treated, local anesthesia may be adequate. For larger areas, sedation or general anesthesia may be recommended. Again, the post-procedure application of various masks, lotions, and/or solutions, typically containing plant extracts and/or other natural or synthetic compounds or chemicals that promote healing and collagen generation is possible.

Laser resurfacing directs short, concentrated pulsating beams of light at irregular skin, precisely removing the skin layer by layer. The two types of lasers most commonly used in laser resurfacing are carbon dioxide (CO2) and erbium. Each laser vaporizes skin cells damaged at the surface-level.

CO2 laser resurfacing, and fractionated CO2 laser resurfacing, use very short-pulsed light energy or continuous light energy that is delivered in a scanning pattern to remove thin layers of the skin with minimal heat damage. Erbium laser resurfacing is designed to remove surface-level and moderately deep lines and wrinkles on the face, hands, neck, and/or chest with even less heat damage, and, therefore, fewer side effects and shorter recovery times.

What is still needed in the art, however, regardless of the skin resurfacing procedure used is an improved post-procedure treatment solution that promotes healing and collagen generation, making the skin healthier and more youthful in appearance.

### BRIEF SUMMARY OF THE INVENTION

In various exemplary embodiments, the present invention provides such an improved post-procedure treatment solution that promotes quicker healing and collagen generation, making the skin healthier and more youthful in appearance. Specifically, the skin treatment protocol of the present invention incorporates an amniotic solution.

Amniotic membrane has been used in the treatment of wounds and the like for many decades, beginning with the application of amniotic membrane obtained from labor and delivery to burns and wounds. Amniotic membrane is rich in collagen and various growth factors that support the healing process. This improves wound closure and reduces scar formation. The lack of immunologic markers in amniotic membrane makes associated allografts perfect for such use. They are also antibacterial and reduce pain on application. In addition, amniotic membrane has been shown to stimulate stem cells to migrate to an area of application. Techniques have been developed to dehydrate amniotic membrane and form a powder (which may then be used to form a solution) while preserving many of its advantageous characteristics. Using these techniques and exploiting these advantageous characteristics, the present invention uses an amniotic solution as part of a post-skin resurfacing treatment, promoting healing and collagen generation, making the skin healthier and more youthful in appearance. The skin is first treated using a skin resurfacing procedure, such as a chemical, dermabrasion, micro-needling, or laser procedure, and subsequently treated with an amniotic solution manufactured from amniotic membrane.

In one exemplary embodiment, the present invention provides a skin treatment method, including: performing a skin treatment procedure on the skin of a patient, wherein the skin treatment procedure includes one of a chemical peel procedure, a dermabrasion procedure, a micro-needling procedure, and a laser procedure; and subsequently, applying a preparation including one or more of amniotic fluid and amniotic membrane material to the treated skin of the patient to promote healing and collagen rejuvenation, wherein the preparation including the one or more of amniotic fluid and amniotic membrane material is prepared from one or more of amniotic fluid, a concentrated amniotic membrane solution, and an amniotic membrane powder. The preparation comprising the one or more of amniotic fluid and amniotic membrane material also includes sterile water or saline. The treated skin of the patent preferably includes one or more of the skin of the face and the skin of the neck.

### BRIEF DESCRIPTION OF THE DRAWING(S)

The present invention is illustrated and described herein with reference to the drawing(s), in which like reference numbers are used to denote like protocol steps/kit components, as appropriate, and in which:

FIG. 1 is a flowchart illustrating one exemplary embodiment of the skin treatment protocol of the present invention.

### DETAILED DESCRIPTION OF THE INVENTION

Again, a variety of medical/cosmetic skin treatment procedures exist for modifying the surface and quality of the skin, especially that of the face and neck. These skin treatment procedures may be performed in a medical office, a pseudo-medical office, and/or a spa or salon.

Aging, genetics, sun exposure, and lifestyle—including nutrition, smoking, and alcohol consumption may all contribute to facial wrinkling, discoloration, and collagen loss. Such conditions may be treated using a skin resurfacing procedure. Skin resurfacing procedures all involve “injuring” the skin and allowing a healing process to take place, resulting in healthier looking skin emerging. This may be enhanced by the post-procedure application of various masks, lotions, and/or solutions, typically containing plant extracts and/or other natural or synthetic compounds or chemicals that promote healing and collagen generation, making the skin healthier and more youthful in appearance. In addition, some are applying PRP derived from a patient's own blood.

Chemical peels involve the application of a caustic solution, dermabrasion utilizes a high-speed abrasive rotary wheel or micro-needles, and laser resurfacing utilizes a laser beam to “injure” the skin.

Chemicals peels may use mild chemicals, such as glycolic acid or the like, to create very superficial changes in the skin, or more harsh chemicals, such as phenol or the like, to create more profound changes in the skin. As the potential for dramatic changes increases, so does the potential risk of permanent skin lightening or scarring. The chemicals are applied to the skin in the desired areas, and may be “neutralized” after an appropriate amount of time. The depth of the peeling action of the chemicals is determined by which chemicals are used, how long they remain on the skin, and whether they are applied lightly or rubbed more vigorously into the skin.

Chemical peels using alpha hydroxy acids (AHAs), such as glycolic acid or the like are the most mild. A single treatment with an AHA gives the skin a fresher, healthier appearance and a radiant glow. Repeated treatments may help to further improve the texture of the skin. No anesthesia or sedation is needed for this type of chemical peel, which produces only a mild tingling or stinging sensation. The post-procedure application of various masks, lotions, and/or solutions, typically containing plant extracts and/or other natural or synthetic compounds or chemicals that promote healing and collagen generation is possible.

Chemical peels using trichloracetic acid (TCA) are also common for treating sun damage. Milder chemical peels using TCA may be repeated frequently in order to achieve cumulative effects, or may be used to achieve a medium or deep chemical peel, depending on the acid concentration and manner of application. Again, the post-procedure application of various masks, lotions, and/or solutions, typically containing plant extracts and/or other natural or synthetic compounds or chemicals that promote healing and collagen generation is possible.

Chemical peels using phenol are common for treating particularly rough and sun-damaged facial skin, for example. However, phenol may cause excessive damage to the skin and result in bleaching, and is typically not used on the neck or other parts of the body. Phenol buffers may be used to create a “buffered” or milder solution, and may allow for greater flexibility in its use. Again, the post-procedure application of various masks, lotions, and/or solutions, typically containing plant extracts and/or other natural or synthetic compounds or chemicals that promote healing and collagen generation is possible.

Dermabrasion uses a high-speed abrasive rotary wheel or micro-needles to abrade or puncture the skin, removing its upper layers. Dermabrasion is sometimes selected for the treatment of facial scars and the like. Dermabrasion often requires some form of anesthesia, whether local or sedation. Again, the post-procedure application of various masks, lotions, and/or solutions, typically containing plant extracts and/or other natural or synthetic compounds or chemicals that promote healing and collagen generation is possible.

Laser resurfacing either creates a uniform injury to the skin, similar to a deeper chemical peel or dermabrasion, or “drills” tiny holes into the deeper layers of the skin in “fractional resurfacing,” where collagen contracts between the tiny holes. Laser resurfacing typically requires anesthesia. Depending on the laser treatment chosen and the area treated, local anesthesia may be adequate. For larger areas, sedation or general anesthesia may be recommended. Again, the post-procedure application of various masks, lotions, and/or solutions, typically containing plant extracts and/or other natural or synthetic compounds or chemicals that promote healing and collagen generation is possible.

Laser resurfacing directs short, concentrated pulsating beams of light at irregular skin, precisely removing the skin layer by layer. The two types of lasers most commonly used in laser resurfacing are carbon dioxide (CO2) and erbium. Each laser vaporizes skin cells damaged at the surface-level.

CO2 laser resurfacing, and fractionated CO2 laser resurfacing, use very short-pulsed light energy or continuous light energy that is delivered in a scanning pattern to remove thin layers of the skin with minimal heat damage. Erbium laser resurfacing is designed to remove surface-level and moderately deep lines and wrinkles on the face, hands, neck, and/or chest with even less heat damage, and, therefore, fewer side effects and shorter recovery times.

The present invention provides an improved post-procedure treatment solution that promotes healing and collagen generation, making the skin healthier and more youthful in appearance. Specifically, the skin treatment protocol of the present invention incorporates an amniotic solution.

Amniotic membrane has been used in the treatment of wounds and the like for many decades, beginning with the application of amniotic membrane obtained from labor and delivery to burns and wounds. Amniotic membrane is rich in collagen and various growth factors that support the healing process. This improves wound closure and reduces scar formation. The lack of immunologic markers in amniotic membrane makes associated allografts perfect for such use. They are also antibacterial and reduce pain on application. In addition, amniotic membrane has been shown to stimulate stem cells to migrate to an area of application. Techniques have been developed to dehydrate amniotic membrane and form a powder (which may then be used to form a solution) while preserving many of its advantageous characteristics. Using these techniques and exploiting these advantageous characteristics, the present invention uses an amniotic solution as part of a post-skin resurfacing treatment, promoting healing and collagen generation, making the skin healthier and more youthful in appearance. The skin is first treated using a skin resurfacing procedure, such as a chemical, dermabrasion, micro-needling, or laser procedure, and subsequently treated with an amniotic solution derived from the placenta or manufactured from amniotic membrane.

Subsequent to the skin resurfacing treatment, such as a micro-needling or laser procedure, the amniotic solution, which is either provided as a fluid or reconstituted from powder or microparticles) is placed on the resurfaced skin.

The items in the treatment kit, used by a technician or physician, include, by way of example only:
Amniotic Solution (0.5 ml, 1 ml, 2 ml, etc.)
10-20 cc Sterile Water or Saline 0.09%
Small Mixing Cup
Sterile Gloves (powder-free)
Sterile Brush, Atomizer/Mister, Dropper, Large Syringe, etc.

The amniotic solution is either: a) reconstituted in a small bowl or the like, if a dry powder/particle preparation is used, where the amniotic solution is 0.5 ml or larger in size and is reconstituted before application, for example, and the total solution used is approximately 10-20 cc once reconstituted with sterile water or saline, or b) if using amniotic fluid, 0.5 ml or larger may be used and no reconstituting is required as the material is prepared following the guidelines specific to that fluid (which may or may not be cryopreserved, requiring proper time to thaw after removal from the freezer).

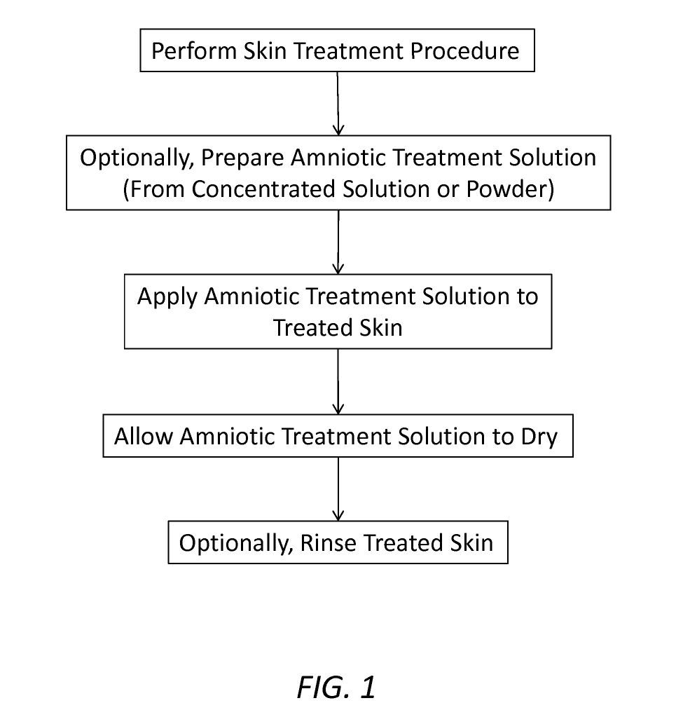

After completion of the skin resurfacing procedure, the technician or physician puts on the sterile gloves and prepares the amniotic solution. Immediately after preparation, the amniotic solution is applied to the face or neck or both using the sterile brush, atomizer/mister, dropper, large syringe, or the like, insuring coverage of the areas treated in the skin resurfacing procedure. The patient sits still until his or her skin dries completely. The patient waits approximately 48 hours, at a minimum, before washing his or her face and avoids soap/cleansers for at least one week. The full process is illustrated in FIG. 1.

Although the present invention is illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A skin treatment method, comprising:
performing a skin treatment procedure on the skin of a patient; and
subsequently, applying a preparation comprising one or more of amniotic fluid and amniotic membrane material to the treated skin of the patient to promote healing and collagen rejuvenation.

2. The skin treatment method of claim 1, wherein the skin treatment procedure comprises one of a chemical peel procedure, a dermabrasion procedure, a micro-needling procedure, and a laser procedure.

3. The skin treatment method of claim 1, wherein the preparation comprising the one or more of amniotic fluid and amniotic membrane material is prepared from one or more of amniotic fluid, a concentrated amniotic membrane solution, and an amniotic membrane powder.

4. The skin treatment method of claim 1, wherein the preparation comprising the one or more of amniotic fluid and amniotic membrane material comprises sterile water or saline.

5. The skin treatment method of claim 1, wherein the treated skin of the patent comprises one or more of the skin of the face and the skin of the neck.

6. A skin treatment method, comprising:
performing a skin treatment procedure on the skin of a patient, wherein the skin treatment procedure comprises one of a chemical peel procedure, a dermabrasion procedure, a micro-needling procedure, and a laser procedure; and
subsequently, applying a preparation comprising one or more of amniotic fluid and amniotic membrane material to the treated skin of the patient to promote healing and collagen rejuvenation, wherein the preparation comprising the one or more of amniotic fluid and amniotic membrane material is prepared from one or more of amniotic fluid, a concentrated amniotic membrane solution, and an amniotic membrane powder.

7. The skin treatment method of claim 6, wherein the preparation comprising the one or more of amniotic fluid and amniotic membrane material comprises sterile water or saline.

8. The skin treatment method of claim 6, wherein the treated skin of the patent comprises one or more of the skin of the face and the skin of the neck.
